(19) United States
(12) Patent Application Publication
(10) Pub. No.: US 2009/0087892 A1
(43) Pub. Date: Apr. 2, 2009

(54) METHODS FOR PRODUCING MUTANT MICROBES USEFUL FOR PRECIOUS METAL AND BIOENERGY PRODUCTION

(75) Inventors: Joe E. Champion, Washington, UT (US); Raymond Owyang, San Francisco, CA (US)

Correspondence Address: RAYMOND OWYANG, 20 Wood Street, SAN FRANCISCO, CA, 94118, US

(73) Assignee: BIOMETAL L.L.C., SAN FRANCISCO, CA (US)

(21) Appl. No.: 12/202,435

(22) Filed: Sep. 2, 2008

Related U.S. Application Data
(63) Continuation-in-part of application No. 11/862,424, filed on Sep. 27, 2007.

(51) Int. Cl. C12P 3/00; C22B 9/00; B22F 9/16; C10G 32/00; C12N 1/19; C12N 15/01

(52) U.S. Cl. 435/168; 75/711; 75/345; 435/254.21; 435/441; 435/281

(57) ABSTRACT

A mutant microbe that generates trace amounts of gold on silver, and uses of the mutant microbe for producing and recovering precious metals and for producing biofuels and oil products from biomass and sedimentary organic matter are described. According to an exemplary embodiment, the mutant microbe is produced by placing metallic silver in an aqueous solution, and adding a species of Saccharomyces to the aqueous solution. When the species of Saccharomyces comes in contact with the metallic silver, at least a portion of the species of Saccharomyces transforms into the mutant microbe that interacts with the metallic silver to form a layer comprising a trace amount of nano gold particles on the metallic silver.

### CROSS-REFERENCE TO RELATED APPLICATIONS

This application is a continuation-in-part of U.S. patent application Ser. No. 11/862,424, filed Sep. 27, 2007, the disclosure of which is incorporated herein by reference in its entirety.

### COPYRIGHT NOTICE

A portion of the disclosure of this patent document contains material which is subject to copyright protection. The copyright owner has no objection to the facsimile reproduction by anyone of the Patent and Trademark Office patent file or records, but otherwise reserves all copyright rights whatsoever.

### FIELD OF THE INVENTION

The present invention relates to methods of mutation of yeast of the genus Saccharomyces with metallic silver and nano silver atoms. The mutant microbes carry out biological transmutation in coating silver with a yellow material comprising trace amounts of nano gold particles. The mutant microbes are useful in a number of applications including the production and recovery of precious metals from mineral ores and the production of biofuels and oil products using both inorganic and organic matter as nutrient sources. The mutant microbes and yeast of the genus Saccharomyces are also useful for aggregating and coalescing nano precious metal atoms into clusters of bulk precious metals where the nano atoms are produced by the resonance of an aluminum or a silver tube in an electromagnetic field.

### BACKGROUND

Biological Transmutation. Biological transmutation can be defined as a nuclear transmutation occurring in living organisms. The phenomenon is not accepted by mainstream science, which argues that transmutations are only possible in high-energy nuclear reactions. Such reactions are physically impossible in biological systems, as the amount of energy used in such a manner would be fatal within a several-kilometer radius. Proponents respond that evidence shows that transmutations do occur, and that the lack of a theoretical model adequately explaining the mechanisms involved (that is, without the emission of deadly amounts of energy) does not render that evidence invalid. The most prominent defender of the existence of biological transmutations is the French scientist Corentin Louis Kervran, who investigated discrepancies between the dietary or environmental intake of elements such as calcium, potassium or magnesium by various organisms and the quantities they hold or excrete. For instance he investigated the source of calcium which chickens use for production of their eggshells, and concluded that they probably convert the calcium from dietary potassium.

Applicants have discovered mutant microbes obtained by treating microbes in aqueous solution with silver and nano silver atoms. The mutant microbes coat metallic bulk silver with a thin layer of a yellow material comprising a trace amount of nano gold particles by a biological transmutation process. Allotropic silver is a yellow-colored metal. But spectroscopic x-ray analysis and conventional metallurgical fire assay methods show the yellow material deposited on silver by the mutant microbes comprises trace amounts of nano gold particles.

Nanotechnology. Nanoparticles are of great scientific interest as they are effectively a bridge between bulk materials and atomic or molecular structures. A bulk material should have constant physical properties regardless of its size, but at the nano-scale this is often not the case.

The properties of materials change as their size approaches the nanoscale and as the percentage of atoms at the surface of a material becomes significant. For bulk materials larger than one micrometer the percentage of atoms at the surface is minuscule relative to the total number of atoms of the material.

Nanoparticles exhibit a number of special properties relative to bulk material. At the nanoscale, matter behaves differently than it does at the bulk scale (>100 nm). A metal's color, melting point, strength, conductivity, magnetism, and crystal structure can be drastically different at the nanoscale. Nanoparticles often have unexpected visible properties because they are small enough to confine their electrons and produce quantum effects. For example, gold nanoparticles appear deep red to black in solution and have melting points in the range of 350 C for nano gold particles with a diameter of 2 nm (nanometer) to 600 C (600 degrees centigrade) for nano gold particles with a diameter of 9 nm. See Cortie, M. B, the Weird World of Nanoscale Gold, Gold Bulletin, vol. 37, 2004, pp. 12-19.

British Patent #GB2,219,995A (Dec. 28, 1989) of David Hudson reports that noble metal elements in monoatomic forms are stable and non-metallic, have electron orbital arrangements in the “d”, “s”, and “p” orbitals that bestow upon the monoatomic forms unique electronic, chemical, magnetic and physical properties.

Microbes for Aggregating and Coalescing Nano Gold. Researchers in Australia have uncovered evidence that a bacterial known as Ralstonia metallidurans may accumulate nano gold particles and aggregate them into bulk gold that looks like coral.

Nano Gold Particles Production. Gold salts are readily reduced to elemental nano gold particles, and chemical methods for making old nanoparticles have been known for a long time. In 2002, Gardea-Torresdey, J. L. et al; Nano Lett,; (Communication); 2002; 2(4); 397-401 reported a biologically based process using ordinary alfalfa plants to accumulate very small nano particles of gold. It is important that gold nano particles be benign to human health and the environment. A goal of many scientists is to introduce gold nano particles into humans to fight cancer and other diseases in pure water, food or nutritional supplements. Potentially, the most environmental friendly method for producing nano gold particles for human health would be nano gold atoms produced by safe and non-pathogenic microbes such as baking yeast.

Phonon Resonance. Online publications in 2001 by co-inventor, Joe E Champion, in the area of phonon resonance proposes a mathematical formula that calculates the exact frequency that a known element will resonate at the resonance frequency of another element and theorizes that when a first element is made to vibrate at the resonance frequency of a second element, the first element can be transformed into the second element.

According to Champion the phonon technology for gold production uses a phonon reactor for resonating silver or aluminum in an electromagnetic field of an electric circuit at certain critical temperatures. Silver is resonated at 43.2° C. and an aluminum block is resonated at 302.9° C. According to Champion, aluminum is transformed to gold and silver is transformed to gold in a low energy nuclear reaction. The nano gold atoms are captured in a water bath. Research is ongoing to verify this phenomena.

Microbes for Precious Metal Recovery. The uses of microbes for recovering precious metals from mineral ores are known. Precious metals are frequently occluded, encapsulated, bonded and/or alloyed in mineral ores and are not amenable to conventional recovery methods. For example, gold often occurs as finely disseminated sub-microscopic particles within a refractory sulfide host of pyrite or arsenopyrite. Bio-oxidation is used to liberate the gold occluded within the sulfide host. A number of processes for bio-oxidizing the sulfide minerals are known in the art. One known method of bio-oxidizing the metal sulfides in an ore is to use bacteria, such as Thiobacillus ferrooxidans, sulfolobus, acidianus species and facultative-thermophilic bacteria in a microbial pretreatment.

Microbes for Biofuel Production. The use of microorganisms to produce methane and ethanol from organic matter are known in the art. For example, ethanol for use as fuel and in alcoholic beverages is produced by fermentation of sugar by certain species of yeast (most importantly, Saccharomyces cerevisiae).

Applicants have discovered that mutant microbes obtained by mutating microbes in aqueous solution with metallic silver or nano silver atoms deposit a thin layer of nano gold atoms and particles onto silver by a biological transmutation process.

Applicants have discovered that nano silver and gold atoms are formed during the mutation process and that these nano atoms can be aggregated and recovered as metallic bulk silver and gold.

Applicants have discovered that the mutant microbes produce silver and nano gold particles and that the silver and gold can be recovered from the biomass of dead mutant microbes of the invention.

Applicants have discovered that the mutant microbes aggregate naturally occurring nano precious metal particles in mineral ore into bulk metal.

Applicants have discovered that the mutant microbes of this invention are useful for production of biofuels and oil products from sedimentary organic matter and biomass, including heavy oil.

Applicants have discovered that precious metals are produced by resonating aluminum or silver tubing in an electromagnetic field.

Applicants have discovered that a biodegradable organic medium, including the mutant microbes of this invention and yeast of the genus Saccharomyces, are useful for coalescing and aggregating nano precious metal atoms produced by resonating an aluminum or silver tube in an electromagnetic field.

### SUMMARY OF THE INVENTION

According to an exemplary embodiment, a mutant microbe that generates trace amounts of gold on silver, and uses of the mutant microbe for producing and recovering precious metals and for producing biofuels and oil products from biomass and sedimentary organic matter are described. According to an exemplary embodiment, the mutant microbe is produced by placing metallic silver in an aqueous solution and adding a species of Saccharomyces to the aqueous solution. When the species of Saccharomyces comes in contact with the metallic silver, at least a portion of the species of Saccharomyces transforms into the mutant microbe that interacts with the metallic silver to form a layer comprising a trace amount of nano gold particles on the metallic silver.

According to exemplary embodiments, the mutant microbes are used for recovering nano precious metals atoms from mineral ores by contacting an aqueous solution of the mutant microbes with a mineral ore.

According to other exemplary embodiments, a method for producing oil products and biofuels from a sedimentary organic rock, heavy oil, and/or a biomass comprises contacting the sedimentary organic rock, heavy oil and/or biomass with the mutant microbe in aqueous solution.

In another exemplary embodiment, a method for bioconverting heavy oil to lower viscosity oil comprises contacting the heavy oil with the mutant microbe in aqueous solution.

In another exemplary embodiment, the mutation process and production of precious metals and biofuels is carried out with air flow from a phonon resonance reactor using coiled silver or aluminum tubing.

### BRIEF DESCRIPTION OF THE DRAWINGS

Objects and advantages of the present invention will become apparent to those skilled in the art upon reading this description in conjunction with the accompanying drawings, in which like reference numerals have been used to designate like elements, and in which:

FIG. 1 is a block diagram of an exemplary phonon resonance reactor according to one embodiment;

FIG. 2 is an image generated by a scanning electron microscope (SEM) depicting mutant microbes at 10,000× magnification;

FIG. 3 is an image generated by an SEM depicting mutant microbes at 20,000× magnification;

FIG. 4 is an image generated by a scanning electron microscope (SEM) depicting Silver Granules Coated with Yellow Material at 1000× magnification;

FIG. 5 is an image generated by a scanning electron microscope (SEM) depicting Mineral Ore before Biotreatment at 1000× magnification;

FIG. 6 is an image generated by a scanning electron microscope (SEM) depicting Mineral Ore after Biotreatment at 10,000× magnification; and

FIG. 7 is an image generated by a scanning electron microscope (SEM) depicting Biomass of Dead Mutant Microbes at 1000× magnification.

### DETAILED DESCRIPTION

Silver Mutation. In an exemplary embodiment, the microbes are mutated from industrial microbes by a process which comprises contacting the microbe(s) in an aqueous solution with metallic silver particles, grains, granules, and/or bars, e.g., silver ranging in size from 1 micrometer (micron) particles to silver bars of 10 kilograms or more. Colloidal silver solutions with colloidal silver in aqueous solution ranging in concentration from 1 ppm to 10 ppm and particle sizes from 1 nanometer to 1 micron can be used. However, metallic silver of 1 micron or more in size to silver bars of 10 kilogram or more is preferably used. In one embodiment, silver grains of particle size from 0.1 millimeter to 1 millimeter and silver needles with high surface area, produced by electrolytic refining, from about 10 microns to 200 microns, can be used. The mutation process can also use nano silver atoms produced in situ by resonating a silver or aluminum tube in an electromagnetic field. In another embodiment, the bioreactor for contacting the silver and microbes is made of silver, for example, a silver tank or container of any size or configuration.

Microbes. In the embodiments described, microbes used in the invention are known commercial microbes widely used in industrial microbiology, including those of the genus of Saccharomyces and Schizosaccharomyces. Species of Saccharomyces include the species: S. cerevisiae, S. bayanus, S. boulardii, S. pastorianus, S. uvarum, S. carlsbergensis, S. ellisoidesu, S. exiguus, S. fragilis, S. chevalieri, S. chodati, S. diastaticus and S. rouxii. Species of Schizosaccharomyces is Schizosaccharomyces pombe. Other commonly known and industrial microbes that can be used include Aspergillus niger, Aspergillus orzae, Ashbya gossypii, Streptomyces species, Bacillus thuringiensis, Rhizobium, Bradyrhizobium, Bacillus subtilis, Corynebacterium glutamicum, Leuconostoc mesenteroides, Streptodornase pyogenes, and Thiobacillus ferrooxidans. The genus Saccharomyces is a fungi that can be used. The species are S. cerevisiae and S. carlsbergensis. These yeasts are commercially available and have been used for baking and beer making for thousand of years.

Mutation Conditions. The contacting can be done without agitation, but preferably with mechanical agitation, air agitation (pumping air or oxygen into the aqueous solution) and/or pumping and passing the microbe in an aqueous nutrient solution through columns, tubes and tanks containing metallic silver or constructed with metallic silver. The mutation may be done at temperatures ranging from 20° C. to 90° C. In one embodiment, a temperature ranging from 30° C. 50° C. is used. Natural sunlight can be a heat source in one embodiment.

Nutrients. The aqueous solution may contain sufficient nutrients to support microbial growth. The useful nutrients are both inorganic and organic compounds commonly used to grow and nourish microbes. Inorganic nutrients include nitric acid, ammonium nitrate, ammonium chloride, ammonium sulfate, sodium nitrate, sulfur, sodium sulfide, sodium chloride, sodium bicarbonate, sodium phosphate, potassium phosphate, ferric chloride, calcium chloride, and ammonium phosphate. Organic nutrients include microbes used for mutation, microbial biomass, glucose, dextrose, sodium acetate, amino acids, and purines. Microbial biomass may be dead microbes being used for mutation Vitamins that can be included in the nutrient solution include pyridoxine, pyridoxamine-HCl, riboflavin, thiamine, niacin, pantothenic acid, p-aminobenzoic acid, folic acid, and biotin. Very small amounts of trace elements such as iron, copper, molybdenum and zinc can also be provided in the nutrient solution. Useful nutrients can also be mineral ores used for recovery of metals and sedimentary organic matter and rocks used for liberation of oil products.

In one embodiment, the mutation process is done in an aqueous solution and the microbes being mutated and the biomass from dead microbes are used as nutrients until the mutant microbes reach a density of 5% to 10% or more. The highest mutant microbe population density is obtained with silver with high surface area produced by electrolytic refining and silver grains of from 0.1 to 1 millimeter.

Silver and Ultraviolet Germicidal Irradiation. In another exemplary embodiment, microbes can be mutated by ultraviolet (UV) germicidal irradiation and by a combination of metallic silver and UV irradiation. UV light is electromagnetic radiation with wavelengths shorter than visible light. UV radiation can be separated into various ranges, with near range (less than 280 nm/2800 Angstrom) considered “germicidal UV”. In one embodiment, UV radiation in the range of 280 nm to 390 nm is used. Exposure of microbes to UV irradiation is done with germicidal lamps that emit germicidal UV electromagnetic radiation. Forced flow of air or water can be used to agitate the microbial solution to ensure exposure to the UV radiation. The mutation using UV light may be done at temperatures ranging from 20° C. to 80° C., and preferably at temperatures ranging from 30° C. to 50° C. In a preferred embodiment, the UV irradiation and the silver germicidal mutation processes are done together.

Electromagnetic Field. In another embodiment, the mutation process is conducted in a bioreactor with means to provide an electromagnetic field. The electromagnetic field can be provided by wrapping the bioreactor, such as a beaker, with copper wire, aluminum wire or silver wire and running an AC current of about 5 to 10 amps through the wire. In large bioreactors, for example, 1000 liters to 10000 liters, the microbial solution can be pumped through a glass column wrapped with copper, aluminum or silver wire for current flow. The current can be provided with a variable transformer power supply.

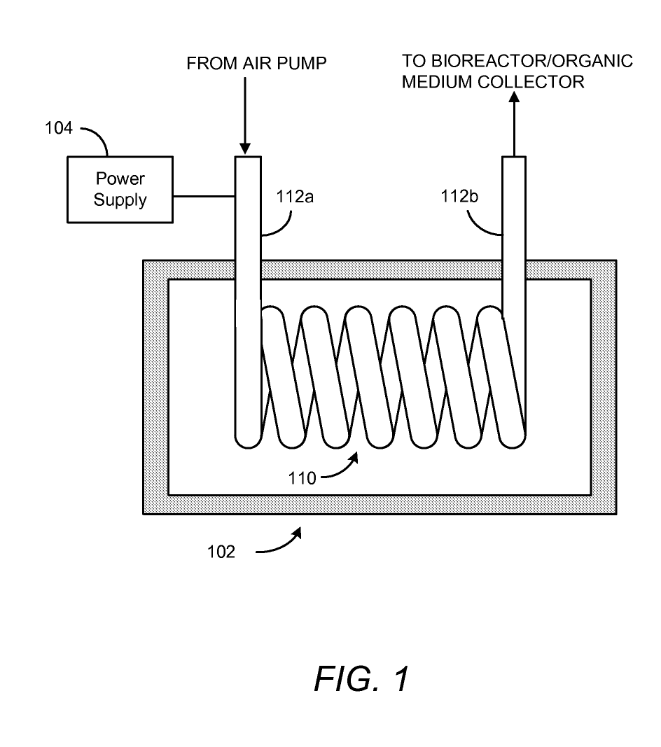

Phonon Resonance Reactor. In another embodiment, the mutation process of contacting silver with a microbe is conducted with air flow from a resonating aluminum or silver tubing carrying an electric charge or from aluminum or silver tubing placed in an electromagnetic field of an electric furnace. Such a furnace, referred to as a phonon resonance reactor, is illustrated in FIG. 1. The silver or aluminum tubing 110 is coiled and placed in the furnace 102. In one embodiment, both ends of the tubing 112a, 112b are passed through holes in the walls or door of the furnace 102. In one embodiment, at least one end of the tubing, e.g., 112a, can be coupled to an external power supply 104 that provides a current through the tubing 110, thereby creating the electromagnetic field.

An air stream is passed through one end, e.g., 112a, and out the other end 112b of the tubing to a bioreactor used for contacting silver with microbes to mutate. Air flow with pressure from 1 psi to 10 psi is passed through to the bioreactor at about 1 ccm (cubic centimeter per minute) to 100 ccm.

In one embodiment, an aquarium-type bubble stone or bubble curtain (not shown) which produces very small bubbles in the range of 1 millimeter or less is used to increase the surface area of the air bubbles flowing to the bioreactor. The tube diameter can range from 1 mm to 2 cm, preferably from 1 mm to 5 mm. The air flow can be passed through the tube clockwise or counterclockwise. The coiled tube 110 can be placed in the furnace 102 with the central axis, i.e., an axis running through the center of each coil, vertical or horizontal. The configuration can also include a plurality of parallel tubes 110 or other common configuration of tubes used for heat exchangers.

For air flow from a silver tube 110, the temperature of the tube 110, and therefore the airflow, can be between 20° C. to 100° C., preferably from 40° C. and 45° C. In one embodiment, the airflow tube 110 temperature is about 43.2° C. For air flow from an aluminum tube, the temperature of the tube 110 and airflow temperature can be between about 20° C. to 350° C., preferably from 270° C. to 310° C. In one embodiment, the temperature is approximately 302.9° C.

It is believed that heating silver to approximately 43.2° C. produces the mechanical vibrations of silver which are the same mechanical vibrational frequencies of gold. Similarly, it is believed that heating aluminum to about 302.9° C. produces the mechanical vibrations of aluminum which are the same as the mechanical vibrational frequencies of gold and silver. The vibrational frequencies in turn assist the microbes in the formation of gold and silver nano atoms and improve and increase the rate of the mutation process.

The mutation process can be conducted at, below or above atmospheric pressure. The mutation can be done in the presence of light or in the absence of light. Light bulbs and sunshine can be used as light sources.

The mutation can be conducted in aerobic or anaerobic conditions. Moreover, the mutation can be conducted in the presence of nitrogen, carbon dioxide, and/or oxygen in the atmosphere. Oxygen can be provided chemically, for example, with hydrogen peroxide, or as a gas from pressurized vessels.

Microbial growth and population density can be measured by conventional direct methods such as plate count, serial dilution, pour plates, spread plates and direct microscope count. Microbial growth can also be measured by indirect methods such as turbidity and metabolic activity. In one embodiment, microbial growth can also be measured by the time period required to coat silver grains of 1 mm to 3 mm with a yellow coating of nano gold particles. With a healthy population of about 5% to 10% or more, the yellow coating is produced in about 2 hours to 24 hours. The yellow coating formed on silver is one test for identifying the mutant microbes of the invention. No other known microbes can coat silver with a yellow colored layer comprised of nano gold particles.

The mutant microbes may be single-celled or multi-celled microbes. They are usually round but can be oval, elongated or flattened on one side. The mutant microbes from the genus Saccharomyces have also been observed to be rod-shaped bacillus. They range from 0.20 to 2.0 micron in diameter and 2 to 8 micron in length. They have been observed to divide by budding and binary cell division. The mutant microbes are also characterized by the ability to stay alive when subjected to high temperatures from 100 C to 150 C, and to strongly basic and acidic environments.

According to one embodiment, the mutant microbes can be identified and characterized by their ability to coat silver granules with a coating of a yellow material which comprises trace amount of nano gold particles. The coating process is done by contacting an aqueous solution of the mutant microbes with metallic silver. The amount of yellow material coated onto the silver ranges from 1 ppm to 1000 ppm and depends upon the contact time, contact temperature and density of the microbial solution. The temperature ranges from 20° C. to 90° C. The contact time ranges from one hour to 100 hours. With a high microbial density of approximately 3 to 5% by weight, the contact time is about 1 to 4 hours. The amount of nano gold particles in the yellow coating is about 100 ppm to 200 ppm or more based on X-ray diffraction analysis and scanning electron microscope analysis.

Once created, the mutant microbes can be stored and maintained by conventional microbiological techniques. A healthy mutant microbe can be isolated and grown to microbe colonies of 1% to 5% by weight in nutrient solutions. Nutrients can be inorganic, including nitric acid, ammonium nitrate, ammonium chloride, ammonium sulfate, sulfur, sodium sulfide, sodium nitrate, sodium chloride, sodium bicarbonate, sodium phosphate, potassium phosphate, ferric chloride, calcium chloride, ammonium phosphate, and organic, including microbes used for mutation, microbial biomass, glucose, dextrose, sodium acetate, amino acids, and purines. Vitamins that can be included in the nutrient solution include pyridoxine, pyridoxamine-HCl, riboflavin, thiamine, niacin, pantothenic acid, p-aminobenzoic acid, folic acid, and biotin. Microbial biomass may be dead microbes being used for mutation. Very small amounts of trace elements such as iron, copper, molybdenum and zinc can also be provided in the nutrient solution. When it is desirable to grow the mutant microbes on a solid medium, a solidifying agent such as agar (a complex polysaccharide derived from a marine alga) is added to the media.

According to one embodiment, the mutant microbes described herein have been found to contain clusters of precious metal atoms within the cytoplasm of the cell. The metals within the cytoplasm are observed as clusters, curved bands and/or circular rings of metal atoms. Some mutant microbes have sets of two to ten concentric rings. Using a scanning electron microscope, the clusters, bands, and/or concentric rings of metal atoms within the cellular structure can be identified as silver and gold atoms and particles.

Mineral Ores. For purposes of this disclosure, the term “mineral” or “mineral ore” means a composition that comprises precious metal values in the form of nano precious metal particles. Thus, a mineral may be a mined mineral, ancient seabed deposit, ancient lakebed deposit, black sands, an ore concentrate, metal bearing sea water, and waste products, such as mining tails, industrial waste water, oil well brine, coal tars, oil shales, tar sands, and oil sands. Useful minerals contain amounts of nano precious metals atoms or particles in the range of 0.1% to 5% by weigh. Generally, the amount of nano particles is only 0.1% to 1% by weight. It is extremely difficult to detect nano precious metal atoms in mineral ores by conventional metallurgical assay procedures such as fire assay, AAS (atomic adsorption spectroscopy), ICP-MS (inductive coupled plasma-mass spectrometer), ICP-AES (atomic emission spectroscopy) and other spectroscopic instrumentation commonly used in analytical laboratories. Thus, the amount of nano metal particles in a mineral ore can only be known after biotreatment with the mutant microbes and the yield of bulk precious metals is determined by recovery. Also, biotreatment with mutant microbes can be used as a diagnostic test for nano precious metal values in mineral ores.

Biomining—Digestion and Nano Metal Recovery. In one embodiment, the digestion and biotreatment of the mutant microbes with the mineral ores are conducted in commercially available bioreactors consisting of a reactor having an agitation means. The agitation means can be mechanical stirring with a flat bladed impeller, percolation column, or air agitated pachuca reactor. The bioreactor can have air intake means, sterilization means, harvesting means, heating and/or cooling means, temperature controller means, pH controller means, filtration means and pressure controller means. All these features of bioreactors are known and commercially available in the biotechnology industry. The digestion the mineral ores by the mutant microbes can also be done by heap leaching techniques. In heap bio leaching techniques, a large body of mineral ore is treated with mutant microbes in nutrient solution in large contaminant ponds with no agitation and/or only occasionally agitation. Generally, the contact time for heap type bio treatment is substantially longer than the agitated bioreactors, and range from 10 days to 100 days.

The mineral ore can be milled and ground to 10 mesh to 300 mesh, preferably 100 to 200 mesh. The minerals useful in the invention are low grade and high grade precious metal minerals containing the nano precious metal particles. Low grade minerals contain from 1 ppb to 1 ppm of a precious metal, preferably gold and silver (1 oz/ton is 34.3 ppm). High grade minerals contain from 2 ppm to 100 ppm. Bio treatment temperature ranges from 15° C. to 50° C., preferably from 20° C. to 30° C. pH can be acidic (pH 1 to 3) or basic (pH 9 to 12), although slightly acidic (pH 4) to slightly basic (pH 8) pH ranges are preferred. The most preferred pH ranges are the neutral range of from pH 6.5 to pH 7.5. At low mutant microbe concentrations, the contact duration is generally longer to allow the mutant microbe to grow and multiply. In one embodiment, the microbe concentration does not exceed the maximum microbe concentration that the nutrient solution can sustain. Contact time can vary from a few hours to several weeks and depends in part on the type and mesh size of the mineral ore digested. Contact time ranges can be from 1 day to 30 days, more preferably from 1 day to 10 days. Generally for ease of agitation, a ratio of mineral ore to microbe/nutrient solution, i.e., the pulp density, varies from 10% by weight mineral ore in the nutrient solution to 50% by weight mineral ore in the nutrient solution.

The digestion can be conducted in aerobic or anaerobic conditions. However, the digestion is preferably conducted in the presence of oxygen, nitrogen and carbon dioxide in the atmosphere. Nutrients can also be provided in the digestion of mineral ore to support growth of the mutant microbes. Nutrients can be inorganic, including nitric acid, sulfur, ammonium nitrate, ammonium chloride, ammonium sulfate, sodium nitrate, sodium chloride, sodium bicarbonate, sodium phosphate, potassium nitrate, potassium phosphate, ferric chloride, calcium chloride, ammonium phosphate, and organic, including the microbes used for mutation, glucose, dextrose, sodium acetate, amino acids, and purines. Vitamins that can be included in the nutrient solution include pyridoxine, pyridoxamine-HCl, riboflavin, thiamine, niacin, pantothenic acid, p-aminobenzoic acid, folic acid, and biotin. Very small amounts of trace elements such as iron, copper, molybdenum and zinc can also be provided in the nutrient solution. In one embodiment, the organic nutrients are present in the mineral ores as sedimentary organic matter. Examples are sedimentary organic rocks such as oil shale, tar sands and other fossil fuels such as anthracite coal, bituminous coal, sub-bituminous coal, and lignite. In one embodiment, inorganic nutrients are present in the metal mineral ores for liberation of metals.

Nutrients, including additional microbes used for mutation, can be added to the digestion as needed to maintain the sufficient mutant microbes for microbial growth and nano metal aggregation. Microbial growth can be measured by conventional direct methods such as plate count, serial dilution, pour plates, spread plates and direct microscope count. Microbial growth can also be measured by indirect methods such as turbidity and metabolic activity.

After digestion with the mutant microbes, the recovery of metal from the mineral ore and microbial solution can be carried out by conventional metallurgical methods such as gravity concentration, smelting, leaching, electrolysis, resins and other methods known to those skilled in art of metallurgy. The nano silver and gold atoms produced in the bioreactor are generally coalesced and aggregated into metal particles which drop to the bottom of the bioreactor and are recovered by decanting. panning or conventional gravity methods of precious methods such vibrating tables, sluice boxes, spiral gold panning bowls, or trommels commonly used in the placer mining industry for the separation and recovery of precious methods.

In another embodiment, the precious metals in the mutant microbes or biomass of dead mutant microbes can be recovered from the biomass of dead mutant microbes. In this embodiment, the precious metals in the biomass of dead mutant microbes can be recovered by burning off the biomass at a temperature of 150° C. to 350° C. to produce a residue of silver and gold, which can be recovered by conventional metallurgical methods.

Sedimentary Organic Matter and Rocks. According to other exemplary embodiments, the mutant microbes can be used for producing oil products and biofuels from sedimentary organic matter and rocks. Suitable sedimentary organic matter includes coal, bituminous coal, sub-bituminous coal, lignite, bitumen, coal tar, fly ash, oil shale, tar sands and oil sands. Sedimentary organic matter that contains a high content of sulfur and sulfides can be used. Oil shale is found in the Western United States, especially the states of Utah, Wyoming, and Colorado, and oil sands found in northern Alberta, Canada. Oil shale is a general term applied to a group of rocks rich enough in organic matter (called kerogen) to yield oil products upon distillation. Oil sands, also referred to as tar sands or bituminous sands, are a combination of clay, sand, water and bitumen. Sedimentary organic matter and rocks generally contain from 1% to 99% organic matter, preferably 10 to 90% organic matter.

Minerals for Metals and Biofuels Co-Production. With sedimentary organic matter and fossil fuels containing precious and base metals, both metals and gaseous and liquid petroleum and metals can be liberated and produced by the mutant microbes. After bio treatment, petroleum products are recovered and refined by conventional petroleum processes and the precious and base metals are recovered by conventional precious metal beneficiation processes such as gravity concentration, amalgamation, electrowinning, cyanidation, etc. In one embodiment, the mutant microbes can be used to treat a mixture of a metal mineral ore and sedimentary organic matter and/or a fossil fuel. In this embodiment, the liquid oil products released from the sedimentary organic matter captures and floats the metals released from the metal mineral ore by a process similar to flotation or froth flotation processes used in the mining industry. The bio treatment procedures used for bio-energy and bio-fuel production are the same as the biotreatment and digestion procedures used for liberation of metals, and are known industrial biotech processing procedures.

Biofuels from Biomass. According to other exemplary embodiments, the mutant microbes can be used for producing oil products and biofuels from biomass. Biomass is any recently living organisms or their metabolic by products. Biomass can be of plant or animal origin. Useful biomass include agricultural residues such as rice straw, stover, wheat straw; agricultural wastes such as sugarcane bagasse, rice hulls, corn fiber, sugar beet pulp, citrus pulp, citrus peels; forestry wastes such as hardwood and softwood thinning and hardwood and softwood residues from timber operations; and wood wastes such as saw mill waste and pulp and paper mill waste; urban wastes such as the paper fraction of municipal solid waste; urban wood waste and urban green waste, and dedicated crops such as switchgrass, hybrid poplar wood, grains, maiden grass. Simple sugars or monosaccharides, such as glucose, fructose, and dextrose can also be used. Preferred biomass feed stocks are plant cellulosic biomass—that is, biomass composed primarily of inedible plant fibers having cellulose and hemicellulose as a prominent component. The biofuel produced depends on the biomass feedstock, and include methanol, ethanol, propanol, butanol, mixed alcohols, and other biogases. In biorefining and bioconverting embodiments, the mutant microbes are used to convert, refine and degrade heavy oils, bitumen, asphalt and tar to lower molecular weight and density petroleum products.

Heavy Oil and Enhanced Oil Recovery. A preferred form of biomass is heavy oil. The mutant microbes can be used for bioconversion, biorefining and biodegradation of heavy oil that is too viscous to ship through a pipeline to lighter oil that can be shipped in pipelines. Examples are surface heavy oil deposits, heavy oil recovered from oil sands and oil shale and heavy oil in oil wells and in depleted and abandoned oil wells. The mutant microbes can be injected into the oil wells with water and/or steam commonly used for secondary and enhanced oil recovery. Once the heavy oil is biodegraded to oil of a lower viscosity, it can be pumped from the oil well and transported in pipelines. The mutant microbes can also be used to bioconvert surface heavy oil deposits to lighter oil products that can also be shipped in pipelines.

Biorefining, bioconversion and biodegrading methods. The digestion and biotreatment of the mutant microbes with biomass, heavy oil and sedimentary organic matter can be conducted in commercially available bioreactors consisting of a reactor having an agitation means. The agitation means can be mechanical stirring with a flat bladed impeller, percolation column, air agitated Pachuca reactors, and continuous flow stirred tank reactors. The bioreactor can have air intake means, sterilization means, harvesting means, heating and/or cooling means, temperature controller means, pH controller means, filtration means and pressure controller means. All these features of bioreactors are known and commercially available in the biotechnology, biorefining and biomining industry. The digestion the biomass and sedimentary organic matter by the mutant microbes can also be done by heap leaching techniques. In heap bio leaching techniques, a large body of mineral ore is placed in a heap or dump where is it irrigated and treated with mutant microbes. Generally, the contact time for heap type bio treatment is substantially longer than the agitated bioreactors, and range from 10 days to 100 days.

The biomass and sedimentary organic matter can be milled and ground to particles in the range of 10 mesh to 300 mesh, preferably 100 to 200 mesh. Bio treatment temperature ranges from 15° C. to 90° C., preferably from 20° C. to 50° C. pH can be acidic (pH 1 to 3) or basic (pH 9 to 12), although slightly acidic (pH 4) to slightly basic (pH 8) pH ranges are preferred. The most preferred pH ranges are the neutral range of from pH 6.5 to pH 7.5.

At low mutant microbe concentration, the contact duration is generally longer to allow the mutant microbes to grow and multiply. Contact time can vary from a few hours to several weeks and depends in part on the type and mesh size of the biomass and sedimentary organic matter digested. Contact time ranges can be from 1 day to 30 days, more preferably from 1 day to 10 days. The ratio of biomass and sedimentary organic matter to microbial solution can vary. Generally for ease of agitation in stirred-tanks, the pulp density can vary from 10% by weight mineral ore in the microbial solution to 50% by weight mineral ore in the microbial solution, preferably about 15% by weight to 25% by weight. The digestion can be conducted in aerobic or anaerobic conditions. However, the mutation is preferably conducted in the presence of oxygen, nitrogen and carbon dioxide in the atmosphere.

Nutrients can also be provided in the digestion of biomass, heavy oil or sedimentary organic matter to support growth of the mutant microbes. Nutrients can be inorganic, including nitric acid, ammonium nitrate, ammonium chloride, ammonium sulfate, sodium chloride, sodium bicarbonate, sodium phosphate, potassium phosphate, ferric chloride, calcium chloride, ammonium phosphate, and organic, including microbes used for mutation, glucose, dextrose, sodium acetate, amino acids, and purines. Suitable media for growing mutant microbes and producing precious metals are nutrient media containing 1 to 10% by weight nitric acid. Vitamins that can be included in the nutrient solution include pyridoxine, pyridoxamine-HCl, riboflavin, thiamine, niacin, pantothenic acid, p-aminobenzoic acid, folic acid, and biotin. Very small amounts of traces elements such as iron, copper, molybdenum and zinc can also be provided in the nutrient solution. The microbes used for mutation, biomass and organic matter present in sedimentary organic matter also serve as nutrients.

Nutrients can be added to the digestion as needed to maintain the sufficient mutant microbes for microbial growth. Microbial growth can be measured by conventional direct methods such as plate count, serial dilution, pour plates, spread plates and direct microscope count. Microbial growth can also be measured by indirect methods such as turbidity and metabolic activity.

Metals and Biofuels Co-Production. With sedimentary organic matter and fossil fuels containing precious and base metals, both metals and gaseous and liquid petroleum and metals are liberated and produced by the mutant microbes. After bio treatment, petroleum products are recovered and refined by conventional petroleum processes and the precious and base metals are recovered by conventional precious metal beneficiation processes such as gravitation concentration, amalgamation, electrowinning, cyanidation, etc. In one embodiment the mutant microbes are used to treat a mixture of a metal mineral ore and sedimentary organic matter and/or a fossil fuel. In this embodiment, the liquid oil products released from the sedimentary organic matter captures and floats the metals released from the metal mineral ore by a process similar to flotation or froth flotation processes used in the mining industry. The bio treatment procedures used for bio-energy and bio-fuel production are the same as the biotreatment and digestion procedures used for liberation of metals and are known industrial bio tech processing procedures.

Biotreatment Enhancement with Phonon Resonance Reactor. In another embodiment, the biotreatment and digestion of mineral ores, sedimentary organic matter and biomass is conducted with air flow from a resonating aluminum or silver tube excited with electromagnetic resonance, i.e., a phonon resonance reactor, as shown in FIG. 1. Although it is not known with certainty, it is believed that the resonance of the electric field causes the silver to resonate at the same mechanical vibrational frequencies of gold which assists the microbes in the production of gold and the aggregation of nano metal atoms. Similarly, it is believed that the force of the electric field causes aluminum to resonate at the same mechanical vibrational frequencies of gold and silver. The coiled tube greatly enhances and improves the resonating of the aluminum and silver vibrations due to containment within tube and increases excitation from resonating back and forth within the tube. The gold and silver vibrational frequencies in turn assist the mutant microbes in the formation of gold and silver nano atoms and improve the digestion and biotreatment processes.

Precious Metal Production inside a Resonating Aluminum and Silver Tube. In another embodiment, precious metals are produced by phonon resonance produced inside aluminum and silver tubing in an electromagnetic field. Other types of metal tubing, for example, platinum, palladium, gold, iron, zinc, titanium, copper, and magnesium, can also be used. For precious metal production, the same metal tube as the desired metal product can be used. For example, a gold tube is used to resonate gold to produce gold and a platinum tube is used to resonate platinum to produce platinum. The precious metals are produced as nano atoms which are coalesced and aggregated with a biodegradable organic medium. The phonon resonance inside aluminum or silver tubing is created with force of an electromagnetic field of an electric kiln shown in FIG. 1 or any known means of producing an electromagnetic field from an electrical circuit. The tubing can be placed inside a glass tube wrapped with electrical wire made of copper, aluminum or silver and an electric current passed through the electrical wire. The tubing can be wrapped with insulated electrically conductive wire, such as ceramic coated electric wire, and an electromagnetic field produced by passing a electric current through the wire. The tubing can be coiled and placed in the proximity of any means to provide an electromagnetic field. For example, an electromagnetic field created by wrapping a non-conducting container, such as a glass container, with copper wire, aluminum wire or silver wire and running an electric current of about 5 to 10 amps through the wire. The current can be provided with a variable transformer power supply with a voltage range of 1 to 100 volts. The aluminum or silver tubing temperature will depend upon the amount electric current passed through the aluminum or silver tubing or the amount of electric current in the electric circuit used to produce the electromagnetic field.

Coalescing and Aggregation of Nano Atoms. The biodegradable organic medium includes the mutant microbes, common commercial microorganisms such as yeast from the genus Saccharomyces or a biodegradable organic compound. Preferred organic compounds are soluble or partially soluble in water and have a decomposition and boiling point of 500° F. or less so that the organic compound can be separated and removed by heating and vaporization in an oven and include low molecular weight aliphatic organic compound with oxygen containing functional groups such as hydroxyl, ether, carbonyl, carboxyl, and ester and include common organic compounds such as sugars, citric acid, acetic acid, oils, and fats. The extent of aggregation and the size of the precious metals produced are dependent upon the time period used for aggregation. Generally, metal clusters can be harvested about every 24 hours by gravity separation. However, the clusters of precious metals are large enough to recover by decanting or gravity separation in about 1 to 2 hours. The metal clusters are off-white to black powders and generally contain about 5 to 10% of the organic medium which can be removed by vaporization at about 500° F.

Cupellation and Fire Assaying. In the embodiments described, cupellation is used to separate noble metals such as gold or silver from base metals such as lead. It is often used to assay gold in order to test its purity. Sometimes cupellation is called “fire assaying.” In this process, an alloy or mineral ore consisting of both noble and base metals is placed in a crucible. Flux materials such as borax glass, sodium carbonate, and wheat flour, can be added. This mixture is then melted and allowed to freeze. When solidified, a button consisting of precious metals and lead can be removed from the slag of metal oxides and other materials. After cooling, the metals are placed in a special pot made of bone ash or clay called a cupel. Under high heat, lead turns to litharge, a lead oxide, which is absorbed by the cupel or lost to the atmosphere. At the end of the cupellation process, a button of pure gold and silver remains in the bottom of the cupel. The button is then placed in nitric acid to dissolve the silver, and the remaining gold weighed to determine the gold content present in the material being assayed. Fire assaying and cupellation are described by C. W. Ammen, Recovery and Refining of Precious Metals, second edition 1993, Chapter 12, pp 302-329.

Metals produced by phonon resonance in an electromagnetic field according to the invention can be cupelled by wrapping in a lead sheet and heating in an electric kiln at about 1000 C. Base metals and lead are absorbed into the cupel and the precious metals form a metallic bead on the cupel.

### EXAMPLES

The above embodiments and other objects, features and advantages of this invention will become apparent to those skilled in the art from the following examples and descriptions of the embodiments. The examples are presented to one of ordinary skill in the art to make and use the invention and are provided in the context of a patent application and its requirements. Various modifications to the preferred embodiments and the generic principles and features described herein will be readily apparent to those skilled in the art. Thus, the present invention is not intended to be limited to the embodiments shown, but is to be accorded the widest scope and consistent with the principles and features described herein.

### Example 1

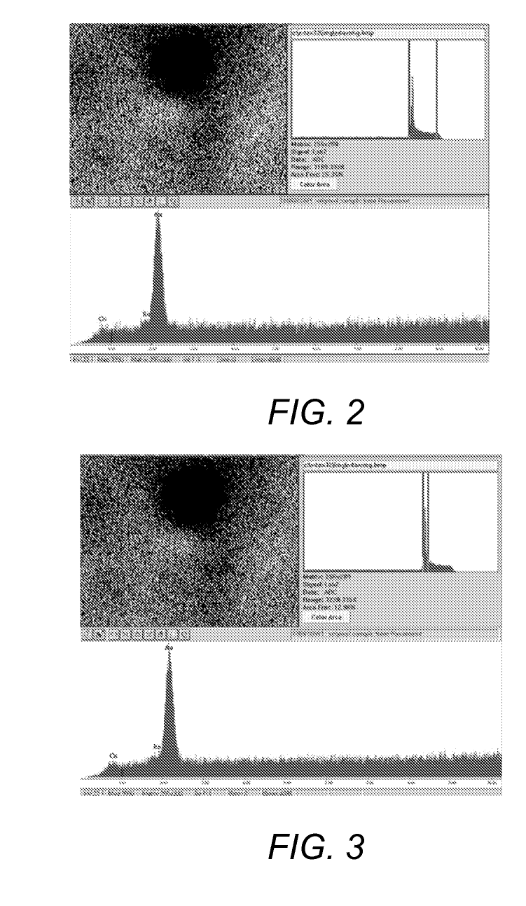

Mutation With Silver Granules. A Petri dish containing 2 grams of silver granules, 4 grams of Saccharomyces cerevisiae and 10 ml of distilled water was stirred one or two times per day over a ten day period. A small sample of aqueous solution was then placed on a glass slide with cover. The slide was then examined under a Meiji binocular biological optical microscope. Live microbes could be observed with a dense band of metal atoms within its cellular structure at magnifications as low as ×500 Some of the live microbes were then examined with a LEO model 1430VP electron scanning microscope with an EDAX x-ray diffraction detector (SEM) at 10,000 and 20,000 magnifications. Clear bands of metal atoms in concentric rings can be observed. Using an EDAX x-ray spectrometer, the metal bands were determined to be metallic. Pictures of the mutant microbe viewed with the SEM are provided in FIG. 2 and FIG. 3.

### Example 2

Mutation in a Silver Container. A 200-ml silver cylindrical container was filled with 100 ml of water and 5 grams of dry active Fleischmann's yeast. The microbial solution was heated at 43° C. on a hot plate for 4 hours. The inside walls of the silver container was coated with a yellow color.

### Example 3

Mutation with Silver in Column. A 200 gallon tank reactor about 2 feet wide, 2 feet deep and 8 feet long was filled with about 600 liters of well water containing trace amounts of naturally occurring minerals. Three kilograms of silver granules was prepared by melting a 99.9% purity silver bars in a gas furnace and pouring the molten silver onto a stainless steel 60 mesh screen placed over a stainless steel drum filled with water. The silver granules were placed in a 6 cm diameter clear plastic percolation column. Five hundred grams of commercially manufactured S. Cerevisiae was added to the tank. The tank reactor was heated to about 40 degrees centigrade (40° C.) with an immersion stainless heater and the aqueous solution was pumped to the top of the percolation column with a submersible pump at the rate of about 40 liters per minute. Water was added as needed to keep the microbial solution volume at about 600 liters. The S. Cerevisiae was allowed to mutate for a period of about 30 days until the population density of mutated microbes reached about 3% to 5% by weight. The mutant microbes in the tank reactor were observed under the SEM to have concentric rings of metal within the cellular structure. Under the optical microscope the mutant microbes appeared were rod shaped with a flatten bottom on one side. The size was about 1 to 10 microns.

After about 10 days, the S. Cerevisiae have been completely mutated and/or died, about 500 grams cane sugar was added as nutrient for the mutant microbes. After about 30 days after most of S. Cerevisiae had been mutated, a 10 gram sample of the microbial solution was dried in a desiccator with a vacuum pump for 18 hours. The residue in the desiccator weighed 0.5 grams.

In addition, the silver in the silver column was emptied and mixed. A ten gram sample of silver granules was removed and the balance returned to the column. The silver granules from the column were a slight to dark yellow color. The ten gram sample was heated in a kiln at about 320° C. After one hour, the yellow-colored metal had vaporized and the silver granules were a silvery color. The silver granules were then cooled and weighed. After heating, the weight of silver granules decreased in weight by 6 mg. Based on this test, the amount of yellow metal coated on the silver granules was in the range of 6 parts in 10,000 parts.

In addition, a 48-gram sample of silver granules coated with the yellow metal from the silver column in the tank reactor was removed after 35 days and placed in an Erlenmeyer flask and with 100 ml of 1N nitric acid solution. The flask was heated on a hot plate until the nitric acid solution reached about 60° C. After about 30 minutes, a small amount of grey-black material was observed in the nitric acid solution and the silver granules no longer had a yellow color. The nitric acid was decanted from the silver granules and filtered onto a filter paper circle placed on a sinister glass filtration unit under vacuum. The nitric acid solution was saved for processing as described below in Example 3D. The filter paper and the grey-black material were washed with distilled water and placed on about 9 grams of a lead sheet, dried, folded and placed into a bone ash cupel. The cupel was heated at 1800° F. for about 30 minutes. On cooling, the cupel had a pale yellow bead weighing about 3 milligrams. The yellow bead was examined on the SEM. The spectrum showed silver and gold peaks.

The silver granules in the Erlenmeyer flask after decanting the nitric acid solution was dried and found to weigh 47.9 grams. The nitric acid solution/filtrate was placed in a beaker and heated to dryness at 200° C. A white and gray residue was formed in the bottom of the beaker. The beaker was then heated to about 280° C. until the white crystals melted to a clear liquid. Distilled water was then added. The grey material which did not dissolve in the water was filtered onto a filter paper circle placed on sinister glass filtration unit under vacuum. The filter paper and grey material were washed with distilled water and placed on about 10 grams of a lead sheet, dried, folded and heated and placed into a bone ash cupel. The cupel was heated at 1800° F. for about 30 minutes. On cooling, the cupel had a pale yellow bead weighing about 3 milligrams. The yellow bead was examined on a scanning electron microscope. The spectrum showed silver and gold peaks. The recovery of precious metals, particularly platinum and palladium, from silver nitrate solutions as described in this Example is commonly called the “Crooks Process” by those skilled in the art of metallurgy.

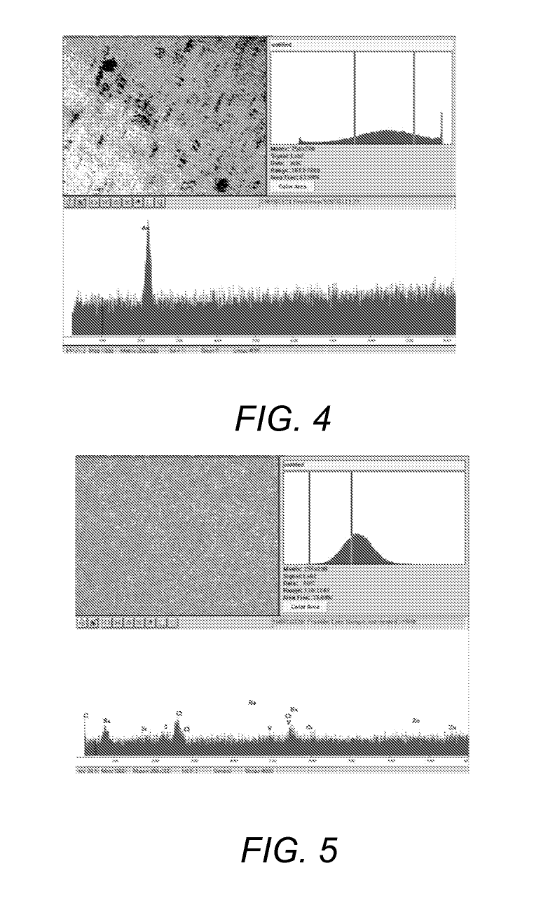

A yellow-colored silver granule from the silver column of was examined with the scanning electron microscope. The spectrum showed a major peak for gold, as shown in FIG. 4.

### Example 4

Silver and UV Mutation. A 37 liter glass tank was filled with 12 liters distilled water, 100 grams of 100 micron to 1 millimeter silver particles and 500 grams of dry active Saccharomyces cerevisiae (Fleischmann brand). The tank was maintained at about 25° C. and agitated with a small fish aquarium pump and an air stone. The tank was exposed to an ultraviolet mercury lamp of 50 watts. After about five to nine days the microbe density was about 3 to 4% by weight.

The mutant microbes were analyzed with an Induced Coupling Plasma-Mass Spectrometer. Small amounts of silver and gold were detected.

Inductive Coupled Plasma (ICP)-Mass Spectrometer (MS) Assays. ICP-MS assays were done on a HP 4500 Series ICP/MS with ShieldTorch System with G1821C Version C.01.01 software.

### Example 5

Silver Mutation with Air Agitation. A 37 liter glass tank was filled with 12 liters distilled water, 100 grams of 100 micron to 1 millimeter silver particles and 1400 grams of the wet form of Saccharomyces cerevisiae (Fleischmann brand). The tank was maintained at about 39° C. degrees centigrade and agitated with an aquarium air pump and an air stone. After about 5 days the microbe density was about 3 to 4% by weight and the silver granules were coated with a thin layer of a yellow material.

### Example 5A

Microbe Growth At High Temperature. A one liter solution of the microbial solution prepared in Example 5 was heated to 95° C. on a hot plate. After two days, the microbial density of the microbial solution was about 3 to 4% by weight and the mutant microbes were moderately active.

### Example 6

Silver Mutation In Salt Water. A 37 liter glass tank was filled with 12 liters distilled water, 100 grams of 100 micron to 1 millimeter silver particles, 1400 grams of the wet form of Saccharomyces cerevisiae (Fleischmann brand) and 12 grams of sea salt. The tank was maintained at about 39° C. and agitated with a 3 psi aquarium air pump and an air stone. After about 7 days the microbe density was about 3 to 4% by weight and the silver granules were coated with a thin layer of a yellow material. The microbes were moderately active.

### Example 7

Colloidal Silver Mutation. A 500 ml beaker was filled with 200 ml of distilled water, 10 grams of Saccharomyces cerevisiae and 50 ml of a 10 ppm colloidal silver solution. The beaker was maintained at about 35° C. and agitated one time every 24 hours with a glass stirring rod. After about 7 days, observation with an optical microscope showed a few live mutant microbes and no live S. cerevisiae. About two grams of silver granules were added to the solution. After about 3 days at a temperature of about 40° C., the silver granules were coated with a pale yellow material.

### Example 8

Mutation with Silver Bars. A ten ounce Engelhard 99.9 silver bar was placed in the tank reactor of Example 3 which uses silver granules in a column. After about ten days, the silver bar was coated a light yellow color with nano gold particles.

### Example 9

Digestion Test Lakebed Ore. A lakebed ore from the Franklin Lake alkali playa, Inyo County, California was used in this test. A 50 g of the ore milled to about 100 mesh, 100 ml of the microbe prepared in Example 3 and 100 ml of distilled water were placed into a 500 ml flat bottom Florence flask. The flask was stirred with a magnetic stir bar and heated to 50° C. for three days. The microbial solution was assayed by the HP 4500 ICP-MS. A two gram sample of the ore residue/solids was placed in aqua regia (one part nitric acid and three parts hydrochloric acid) at about 20° C. The aqua regia solution was analyzed with the HP 4500 ICP-MS. Gold, silver and palladium in the amount of 10 ppm to 100 ppm are detected in the aqua regia solution.

### Example 9A

A sample of the ore used in Example 9 was examined with the Leo 1430VP scanning electron microscope. The spectrum showed no silver and gold peaks. See FIG. 5.

### Example 9B

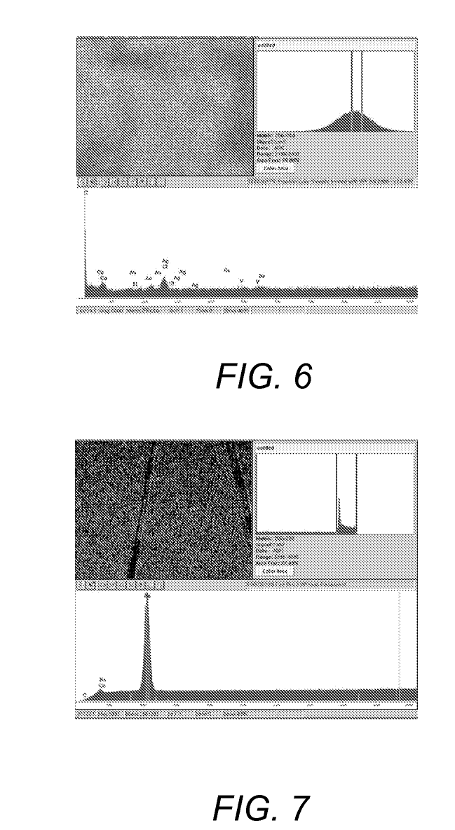

A sample of the ore biotreated in Example 9 for 3 days was dried and examined with the Leo 1430VP scanning electron microscope. The spectrum showed silver and gold peaks. See FIG. 6.

### Example 10

Silver Needles Mutation & Metal Production-A 15-gallon round rubber container about 30 cm high and 60 cm diameter at the top and 40 cm diameter at bottom was filled with 900 grams of Fleischmann's active yeast, 20 liters of water and 400 grams of 150 micron to 250 micron (60×100 mesh) electrolytic silver needles purchased from Academy Corporation, New Mexico, USA. Most of the silver needles used had a smooth surface from being electroplated on a smooth cathode plate in the electrorefining process. The tank was placed on level ground with full sun exposure at St. George, Utah during April to June when the average temperature ranged from 75-80° F. during the day to 35-50° F. during the evening. The container was stirred with a wooden spoon about two to three times a day to thoroughly suspend and disperse the silver through the yeast and water mixture. About every 3 to 4 days, another 450 grams of yeast was added. Water was added as needed to maintain the volume in the container at about 20 liters. After 10 days, about 60 grams silver needles were scooped from the bottom of the container, placed in a beaker and washed with water. The silver needles were a dark yellow color and has the appearance of gold dust.

### Example 10A

Gold in Starting Silver Needles. A 60 gram sample of the dried silver needles of Example 10 was placed in a 250 ml beaker with 100 ml of nitric acid (1 part 70% nitric and 4 parts water). The beaker was warmed on a hot plate to about 50° C. The 60 grams of silver needles dissolved and a trace amount of black sponge was formed. The contents of the beaker were evaporated to dryness on a hot plate at 200° C. The temperature of the hot plate was raised to 280° C. and the residue in the beaker turned a brown color. The temperature was then raised to 325° C. and the residue in the beaker formed a clear molten liquid with a black spongy material. The beaker as cooled to 30° C. and 50 ml of distilled water was added. The beaker was warmed to dissolve any silver nitrate residue in the beaker. The contents of the beaker were then filtered. The filter paper which contained a black material was dried. The black material and dried filter paper were heated in ceramic dish to give 1.4 grams of metallic gold.

### Example 10B

Gold in Microbial Solution. Water was added to the 15-gallon container of Example 10 as needed to keep the volume of the microbial mixture at about 20 liters. About 450 grams of additional yeast was added every 4 to 6 days. The container was stirred about 1 or 2 times a day. After 30 days, the silver needles in the container of Example 10 were allowed to settle to the bottom. A cup of microbial mixture was then taken from the top of the mixture in the container and placed into an aluminum pie pan and dried in an oven at 180° C. for 4 hours to produce brown residue of biomass weighing about 50 grams. The residue was then placed in a scorifying dish and heated in an electric kiln at 260° C. for 3 hours and then at 370 hours for another 3 hours. Black smoke was produced from the residue. After the black smoke stopped, the residue was heated at 1090° C. and kept at this temperature for 30 minutes. On cooling a pale yellow metal bead weighing 21 grams was obtained. The bead dissolved in nitric acid and was primarily silver.

### Example 10C

Nitric Acid Parting. The 21-g metal bead of Example 10B was placed in a 100 ml beaker with 100 ml of nitric acid (1 part 70% nitric and 4 parts water). The beaker was warmed on a hot plate to about 50° C. The bead dissolved and a trace amount of black sponge was formed. The contents of the beaker were evaporated to dryness on a hot plate at 200° C. The temperature of the hot plate was raised to 280° C. and the residue in the beaker turned a brown color. The temperature was then raised to 325° C. and the residue in the beaker formed a clear molten liquid with a black spongy material. The beaker was cooled to 30° C. and 20 ml of distilled water was added. The beaker was warmed to dissolve any silver nitrate residue in the beaker. The contents of the beaker were then filtered. The filter paper which contained a black material was dried. The black material and dried filter paper were heated in a ceramic dish to give 2 grams of metallic gold.

### Example 10D

Removal of Starting Silver. Water was added to the container of Example 10 as needed to keep the volume at about 18 to 20 liters. About 450 grams of yeast was added every 5 to 7 days. The container was manually stirred one to two times a day. After 45 days, the silver needles in the 15-gallon container of Example 10 which was allowed to settle to the bottom of tank. The microbial mixture was decanted into two 5-gallon buckets. Water was used to wash the silver needles on the bottom of container. From the bottom of the container, 290 grams of silver needles which looked like gold dust was recovered. The rinse water used to wash the silver needles was added to the two 5-gallon buckets of microbial solution to give a volume of about 10 liters in each bucket.

### Example 10E

Metal Recovery With Phonon Resonance Unit. The two 5-gallon buckets from Example 10D was placed side by side outdoors in full sunlight and a temperature of 75-85° F. during the day. One of the buckets (bucket #1) from example 10D was treated with air flow from the aluminum phonon resonance reactor described in Example 26 for about 12 hours per day for a period of 15 days. The phonon reactor was operated at 302-305° C. After 15 days, the bucket was shaken with an industrial shaker for about 30 minutes. The shaking caused a metal product to drop to the bottom of the bucket. The microbial solution was decanted from the bucket and the metal product on the bottom of bucket was washed with water and dried to give 120 grams of dark yellow metal product of about 0.1 milliliter to 0.5 milliliter size. Under the 100-power microscope the metal product looked like tiny nuggets without any smooth metallic surfaces found in the starting silver needles. A ten gram sample was wrapped in about 9 grams of lead sheet; the lead sheet was folded and placed onto a 1.5 inch bone ash cupel. The cupel was heated slowly to 1850° F. in an electric kiln and maintained at this temperature for 30 minutes. The cupel was removed from the kiln and cooled. A very pale yellow bead weighing 9.5 grams was obtained. A second bead weighing 0.9 grams was made in the same manner from a 1 gram sample of dark yellow metal product. The 1 gram bead was placed in dilute nitric acid (1 part 70% nitric acid and 4 parts water) at 45-50° C. for about 60 minutes. The bead dissolved to give only about 5 milligrams of black sponge. The addition of sodium chloride to the nitric acid solution gave a thick white precipitate of silver chloride. The HP 4500 ICP-MS showed only silver in the nitric acid solution.

### Example 10F

Metal Recovery Without Phonon Resonance Reactor. After 15 days, the second 5-gallon bucket (bucket #2) from Example 10D was processed as described for bucket #1 in Example 10E, except no air flow from the aluminum phonon resonance reactor was provided. This bucket gave 85 grams of silver product. Under the 100-power microscope the silver product looked like tiny nuggets without any of smooth metallic surfaces found in the starting silver needles. A 10-gram sample was cupelled as described in Example 10E. A 9.5 gram bead was obtained. This bead had the same color as the bead from Example 10E and parted in nitric acid to give silver nitrate. The HP 4500 ICP-MS showed only silver in the nitric acid solution.

### Example 11

Silver Needles Mutation—Into a 5-gallon plastic white bucket were placed 1 kilogram of 60×100 mesh of silver needles, 906 grams of Fleischmann's yeast and 7.5 liters of municipal tap water from Washington County, Utah. The silver and yeast were thoroughly mixed and the bucket was placed on level ground at Washington County, Utah with full exposure to sunlight at a temperature of about 35-50° F. in the evening and 60-80° F. during the day. The bucket contents were mixed with a wooden spoon about two to three times a day. The silver needles became suspended throughout the yeast water mixture. After 2 days, another 453 grams of yeast was added. The water was allowed to slowly evaporate over the next eight days to give a thick biomass of yeast, mutant microbes and silver needles. The contents of bucket weighed about 2.2 kilograms.

### Example 11A

Silver Recovery. The contents of the bucket from Example 11 were transferred into three 5-gallon white plastic buckets. Into one bucket (marked #11A1) was placed ⅓ of the contents of bucket and 3.7 liters of distilled water and 900 grams of Fleischmann's yeast. This bucket was placed in the sunlight during the day at Washington, Utah and stirred with a wooden spoon about 2 to 3 times a day. After three days another 450 grams of yeast and 1.8 liters of water were added. After about another 7 days, another 450 grams of yeast and 1.8 liters of water were added. The silver needles in this bucket were allowed to settle to the bottom of the bucket. A sample of silver needles was scooped from the bottom of the bucket, washed with water to remove most of the organic matter, and then dried to give 8.9 grams of silver needles colored a dark yellow color.

### Example 11B

Nitric Acid Parting and Crooks Process. The 8.9 grams of silver needles recovered from bucket #11A1 as described in Example 11A was placed in a 100 ml beaker with 20 ml of nitric acid (1 part 70% nitric and 4 parts water). The beaker was warmed on a hot plate to about 50° C. The silver needles dissolved and a trace amount of black sponge was formed. The contents of the beaker were evaporated to dryness on a hot plate at 200° C. The temperature of the hot plate was raised to 280° C. and the residue in the beaker was a brown color. The temperature was then raised to 325° C. and the residue in the beaker formed a clear molten liquid with a black spongy material. The beaker was cooled to 30° C. and 20 ml of distilled water was added. The beaker was warmed to dissolve any silver nitrate residue in the beaker. The contents of the beaker was then filter. The filter paper which contained a black material was dried. The black material and dried filter paper were heated in a ceramic dish to give 1.1 grams of metallic gold.

### Example 12

Silver Shots In Black Bucket. Into a 5-gallon plastic black bucket were placed 300 grams of 99.9 purity silver grains of about 1 mm to 3 mm, 906 grams of Fleischmann's yeast and 7.5 liters of municipal tap water from Washington County, Utah. The bucket was covered with a black plastic top, and the contents were thoroughly mixed. The bucket was placed on level ground at Washington County, Utah with full exposure to sunlight at a temperature of about 35-50° F. in the evening and 60-80° F. during the day. The bucket contents were mixed by swirling the bucket about two to three times a day to loosen the silver shots on the bottom of the bucket. After 2 days, another 453 grams of yeast was added. After 4 days, the silver shots were a pale yellow color. After 7 days, the silver shots were a golden yellow color.

### Example 12A

Coiled Silver Phonon Resonance Treatment. After 7 days, the microbial solution in Example 12 was decanted from the silver shots at the bottom of the bucket and transferred to a clean new white plastic bucket. Air flow at 4 psi from the phonon resonance reactor with a silver coil described in Example 27 was passed into the new bucket for 7 days. The phonon resonance reactor temperature was from 40 to 45° C. A small sample of the microbial mixture in the bucket was panned in a gold plastic panning dish about 18 inches in diameter. A small amount of a metallic material with a silvery color about 0.1 to 0.5 mm in size was obtained. After 9 days, the bucket was shaken with an industrial shaker and the metal produced was dropped to the bottom of the bucket. The microbial mixture was decanted from the metal product on the bottom of bucket. The metal product was washed with water and dried to give 71 grams of silvery product. The silver product was melted at 2000° F. in an electric kiln to give 63 of grams of silver metal.

### Example 13

Digestion Test Arizona ore. The mutant microbes prepared by the method of Example 3 was used to digest a gypsiferous mineral ore of red mudstone and siltstone with thin-bedded to laminated gypsum and green mudstone from during the Tertiary period from the Tonto Basin area of Arizona. The digestion procedure was carried out for three days according to the procedure of Example 9. A two gram sample of the ore residue/solids was placed in aqua regia (one part nitric acid and three parts hydrochloric acid) at about 20° C. The aqua regia solution was analyzed with the HP 1500 ICP-MS. Gold, silver and palladium in the amount of 10 ppm to 100 ppm are detected in the aqua regia solution.

### Example 14

Digestion Test-Oil Shale. This test used oil shale from the Green River Formation of Wyoming and Colorado. A 50 g sample of the oil shale (about 100 mesh), 100 ml of the microbe prepared by the method of Example 3 and 100 ml of distilled water were placed into a 500 ml flat bottom Florence flask. The flask was stirred with a magnetic stir bar and heated to 80° C. for three days. The microbial solution was assayed by the HP 4500 ICP-MS and the solution was found to contain about 10 ppm silver.

### Example 15

Digestion Test-Flotation Concentrates of Arsenosulfide Ore. A flotation concentrate having about 30 ppm gold was used in this test. The concentrate was prepared from an arsenosulfide ore from the Shandong Province of China. A 50 g sample of the concentrate, 100 ml of the microbe prepared by the method of Example 3 and 100 ml of distilled water were placed into a 500 ml flat bottom Florence flask. The flask was stirred with a magnetic stir bar and heated to 50° C. for three days. A two gram sample of the ore residue/solids was placed in aqua regia (one part nitric acid and three parts hydrochloric acid) at about 20 C. The aqua regia solution was analyzed with the HP ICP-MS. Trace amounts of silver was detected in the aqua regia solution.

### Example 16

Digestion Test-Flotation Tails. The tails from a flotation concentrate having about 1 ppm gold was used in this test. A 50 g sample of the tails, 100 ml of the microbe prepared by the method of Example 3 and 100 ml of distilled water were placed into a 500 ml flat bottom Florence flask. The flask was stirred with a magnetic stir bar and heated to 50° C. for three days. A one gram sample of the ore residue/solids was placed in aqua regia (one part nitric acid and three parts hydrochloric acid) at about 20° C. Trace amounts of silver was detected with the ICP-MS.

### Example 17

Digestion Test on Vernal Oil Shale. A 500 g (100 mesh) sample of oil shale from Vernal, Utah (Bureau Land Management stockpile for research testing), 1000 ml of mutant microbe solution prepared by the method of Example 3 with about a 3% microbe density by weight was contacted in a 2 liter beaker at a temperature of about 80° C. The digestion mixture was stirred periodically with a glass stirring rod. After six hours, the mixture was allowed to settle. The shale settled to the bottom of beaker. On top of the shale was a thin layer of oil products released from the shale. On top of the oil layer was the aqueous microbial solution. The beaker was stirred periodically for another 48 hours at a temperature of about 80° C. After the additional digestion time, the mixture was allowed to settle. The shale residue settled to the bottom. The next layer was the microbial aqueous solution. The organic layer was on top of the aqueous solution.

### Example 18

Digestion Test on Tar Sands. A 50 g sample of tar sands from the Athabasca deposit in Alberta, Canada and 200 ml of the microbial solution prepared by the method of Example 3 were placed in 500 ml beaker. The beaker was agitated with an aquarium pump and air stone and heated to 60° C. on a hot plate. After about 5 days at 60° C., the tar was released from sands leaving a mixture of light grey sand and tar in the microbial solution. When the beaker was heated at 80° C. for 24 hours, the tar became light oil that floated to the top of the microbial solution.

### Example 19

Metal Recovery from Microbial Solution and Biomass. A 2 ml sample from the tank reactor described in Example 3 was removed after 60 days and placed into a clay scorifying dish and evaporated to dryness at 100° C. The dish was then placed into an electric kiln with tungsten elements and heated to about 320° C. for 14 hours. About 10 grams of lead sheet was added and the dish heated to about 980° C. The molten lead and slag was then poured into a cone mold. The lead was separated and pounded into a cube. The lead cube was placed into a bone ash cupel and heated at 980° C. to give 5 mgs of a bead with a light yellow color. The bead was placed in dilute nitric acid (1 part 70% nitric acid and 4 parts water) at 45-50° C. for about 60 minutes. The bead dissolved to give only a trace of black sponge. The addition of sodium chloride to the nitric acid solution gave a thick white precipitate of silver chloride. The HP 4500 ICP-MS showed silver in the nitric acid solution.

### Example 20

Mutation with Silver Granules. A quart jar with a metal lid was filled with 500 ml of distilled water, 7 grams of Saccharomyces Cerevisiae (Fleischmann's brand) and 10 grams of silver granules of about 1 mm to 5 mm. The jar was loosely covered with the lid and heated on a hot plate to bring the solution temperature to about 35° C. After about 5 days, the silver was coated with a pale yellow metallic material. The observation of the microbial solution with an optical microscope showed that the mutant microbe density was about 1%.

### Example 21

Aerobic Silver Mutation. A second test in a quart jar was done as described in Example 20. All reaction conditions and materials were identical except that an air stone was used to pump air into the bottom of the jar. After about 5 days, the silver was coated a yellow color that was visually observed to be more yellow than the Example 20. Also, the observation of the microbial solution with an optical microscope showed that the mutant microbe density was about 2%.

### Example 22

Mutant Microbe Diagnostic Test. A 500 ml sample of the microbial solution prepared by the method of Example 3 having a mutant microbe density of about 3% was placed in a beaker with 20 grams of silver granules sized about 1 mm to 5 mm. The solution was heated at 39° C. After about 4 hours, the silver was observed to have a yellow coating.

### Example 23

Mutation At Elevated Temperature. Mutation at 80° C. A 500 ml sample of the same microbial solution used in Example 22 was placed in a beaker with 20 grams of silver granules sized about 1 mm to 5 mm. The solution was heated at 80° C. After about two hours, the silver granules were coated with a yellow color.

### Example 24

Mutation in Salt Water. A 500 ml sample of the same microbial solution used in Example 22 was placed in a beaker with ten (10) grams of sea salt. The microbial solution was heated to 90° C. for 24 hours. Observation of the microbial solution after cooling with an optical microscope showed the microbial solution had a mutant microbe density of about 3 percent that was moderately active.

### Example 25

SEM Scan of Microbe Biomass. After 90 days, the content of the microbial tank of Example 3 was evaporated to dryness at about 25° C. to 30° C. over a 90-day period to give a biomass of dead microbes. The biomass was examined with the Leo 1430VP scanning electron microscope. The spectrum shows a major peak for gold. See FIG. 7.

### Example 26

Coiled Aluminum Phonon Resonance Reactor. An 8-feet length of Anderson Barrows ⅜ inch soft aluminum coil tubing purchased at Ace Hardware was coiled in loops of about 2.5 to 3 inch diameter. The coil was installed in an electric kiln, (Vcella Kilns, Model #9) with inside dimensions of 9 inch wide, 10 inch deep and 6.5 inches high. Three small holes were drilled into a wall of the kiln. The ends of the coiled aluminum tube are placed into two of the holes. A temperature probe connected to a model #210 J-KEM Scientific temperature controller was inserted into the third hole. One end of the aluminum tube is connected to a 4 psi air pump. The other end of the coiled tube is connected to an aquarium-type bubble curtain or stone and placed on the bottom of a container (bioreactor) with a biodegradable organic medium.

### Example 27

Coiled Silver Phonon Resonance Reactor. A phonon resonance reactor was constructed as described in Example 26, except that the coiled aluminum tube was replaced with a coiled silver tube made from 2 meters of a 99.95% purity silver tube with 3 mm inside diameter and 3.5 mm outside diameter, manufactured by Goodfellow Corporation, Oakdale, Pa.

The silver phonon resonance unit can also be a coiled silver tube heated in a water bath or other heating means to an operating temperature of 40° C. to 45° C.

### Example 28

Mutation With Silver and Coiled Aluminum Phonon Resonance Reactor. A 20 gallon plastic tank reactor about 2 feet wide, 2 feet deep and 2 feet long was filled with about 10 gallons of distilled water. One kilogram of 99.9% purity silver grains from 1 mm to 5 mm was placed in the bottom of the tank. One kilogram of commercially manufactured S. Cerevisiae (Fleischmann's yeast) was added in about 100-gram portions to the tank. The tank reactor was heated to about 43° C. with an immersion stainless heater. Air flow at 4 psi from an aluminum phonon resonance reactor as described in Example 26 was provided to the tank reactor through a bubble curtain placed on the bottom of the tank. Air flow was heated to 300° C. to 305° C. in the phonon resonance reactor. Every 3 to 4 days another 100 grams of yeast was added to the tank reactor. After 2 days, the silver in the tank turned to a pale yellow color. After 4 days, the silver was a golden yellow color. After 7 days, the silver was a dark yellow color. Water was added as needed to keep the tank reactor volume at about 9 to 10 gallons. After 10 days the microbial solution was pumped into another container and the silver was removed from the bottom of tank reactor.

### Example 29

Vibrational Table Collection. The microbial solution from Example 28 was returned to the tank reactor after the starting silver was removed from the tank. The air flow from the phonon resonance reactor to the tank reactor was kept in operation about 15 hours per day. About 100 grams of yeast was added every 3 to 4 days and water was added as needed to maintain a volume of about 10 gallons. The microbial solution was heated to about 40° C. to 45° C. After about 3 days, a silvery metal product started to drop to the bottom of the tank. The metal product and microbial solution were pumped to a commercial gravitational vibrating concentrating table with a ¼ HP motor, neoprene top and tapered riffles. The table collected a silvery metal product at a rate of about 30 to 50 grams per day. The microbial solution was recycled back to the tank reactor.

### Example 30

Mutant Microbes with In Situ Silver Production. No silver was used in this test and silver for mutation is made in situ. A 20 gallon plastic tank reactor about 2 feet wide, 2 feet deep and 2 feet long was filled with about 10 gallons of distilled water. One kilogram of dry active Fleischmann's yeast was added in 100 grams portions to the tank. The tank reactor was heated to about 43° C. with an immersion stainless heater Air flow at 4 psi from a phonon resonance reactor with a silver coil as described in Example 27 was provided to the tank reactor through a ¼ inch plastic tube a with bubble curtain placed on the bottom of the tank. Air flow was heated to 43° C. in the phonon resonance reactor. Every 3 to 4 days another 100 grams of yeast was added to the tank reactor. Every day a small sample of the microbial solution was panned in a gold plastic panning dish about 18 inches in diameter. A small amount of a metallic material with a silvery color about 0.1 to 0.2 mm in size was obtained after two days. After 5 days, panning of the microbial solution showed a metallic material with a silver color about 0.1 to 0.5 in size. After 9 days, the test with the tank reactor was stopped and the metallic product was allowed to settle for a two day period. The microbial mixture was decanted from the metal product on the bottom of tank. The metal product was washed with water and dried to give 23 grams of metal product. The metal product was melted at 2000 F in an electric kiln to give 115 grams of grams of a silver-colored metal.

### Example 30A

Mutant Microbe Diagnostic Tests. The microbes in the tank reactor of Example 30 were observed under the SEM to have concentric rings of metal within the cellular structure characteristic of the mutant microbes of the invention. Under the optical microscope the mutant microbes appeared were rod shaped with a flatten bottom on one side. No starting yeast was observed in the microbial solution. A 10 gram sample of 99.9% purity silver grains of about 1 mm to 4 mm and 100 ml of the microbial solution were placed in a beaker. The beaker was heated at 43° C. for 10 days. The silver grains had a pale yellow color after two days and a pale to dark yellow color after 5 days.

### Example 31

Yeast Mutation with In Situ Silver from Aluminum Phonon Resonance Reactor. No silver was used in this test and silver for mutation was made in situ. A 20 gallon plastic tank reactor about 2 feet wide, 2 feet deep and 2 feet long was filled with about 10 gallons of distilled water. One kilogram of commercially manufactured S. Cerevisiae (Fleischmann's yeast) was added in 100 grams portions to the tank. The tank reactor was heated to about 43° C. with an immersion stainless heater. Air flow at 4 psi from an aluminum phonon resonance reactor as described in Example 26 was provided to the tank reactor through a bubble curtain placed on the bottom of the tank. Air flow was heated to 300° C. to 305° C. in the phonon resonance reactor. Every 3 to 4 days another 100 grams of yeast was added to the tank reactor. Water was added as needed to keep the tank reactor volume at about 9 to 10 gallons. Every day a small sample of the microbial solution was panned in a gold plastic panning dish about 18 inches in diameter. A small amount of a very fine metallic material with a silvery color about 0.1 mm or less was observed after about 8 hours. After two days, panning of the microbial solution showed a silver colored metal about 0.1 mm to 0.2 mm. After 5 days, panning of the microbial solution showed metallic material with a silver color about 0.1 mm to 0.5 mm in size.

### Example 31A

Collection with Spiral Gold Panning Wheel. After 9 days of operation, the collection of the metal product in microbial mixture of Example 31 was started using a model GRAC vibrating spiral gold panner, manufactured by Keene Engineering, Chatsworth, Calif. The machine comprises a rotating 24 inch wheel with undercut riffles that vibrates up to 100 times per minute. The wheel was set at an angel of 45 degrees. The microbial mixture was pumped to the bottom of edge of the wheel and the microbial solution is recycled to the tank reactor of Example 31. The metal product was collected as it vibrates to the center of wheel. The wheel was operated about 2 hours per day and collected about 30 grams of a silvery metal product per day. The SEM showed that the product was primarily silver with about 1% gold, 0.5% platinum and 0.5% palladium.

### Example 32

Oil Shale with Microbes and Coiled Aluminum Phonon Resonance Reactor. A 20 gallon plastic tank reactor about 2 feet wide, 2 feet deep and 2 feet long was filled 5 kilograms of oil shale from Vernal, Utah that was milled to about 40 to 50 mesh and with 10 gallons of mutant microbes with a microbial density of about 5%, prepared as described in Example 28. One kg of dry active Fleischmann's yeast was added in 100 grams portions to the tank. The tank reactor was heated to about 43 degrees centigrade with an immersion stainless heater Air flow at 4 psi from a phonon resonance reactor with an aluminum coil as described in Example 26 was provided to the tank reactor through a bubble curtain placed on the bottom of the tank. Air flow was heated to 302° C. to 305° C. in the phonon resonance reactor. After one day, a thin oil layer was observed on top of the microbial solution. Every 3 days, a small sample of oil shale was scooped from the tank and panned in a gold plastic panning dish about 12 inches in diameter. A small amount of a metallic material with a silvery color about 0.1 to 0.2 mm in size was separated from the oil shale. After 5 days, the oil shale was scooped from the tank and processed in the spiral gold panner described in Example 31A. A silver-gray powder about 0.1 to 0.5 in size was collected. The microbial solution was recycled to the tank reactor and oil shale was removed from the collection tank of the spiral wheel as a waste product. After all the oil shale had been processed in the spiral wheel, about 30 grams of an oil product was separated by from the oil layer by skimming the top of microbial solution and oil layer into a 1 liter separatory funnel.

### Example 33

Oil Shale with Coiled Aluminum Resonance Reactor. A 20 gallon plastic tank reactor about 2 feet wide, 2 feet deep and 2 feet long was filled 5 kilograms of oil shale from Vernal, Utah that was milled to about 40 to 50 mesh and 10 gallons of water. The tank reactor was heated to about 50° C. with an immersion stainless heater Air flow at 4 psi from a phonon resonance reactor with an aluminum coil as described in Example 26 was provided to the tank reactor through a bubble curtain placed on the bottom of the tank. Air flow was heated to 302° C. to 305° C. in the phonon resonance reactor. After 8 hours, a few drops of oil were observed on the surface of the oil shale. After two days, a thin layer of oil was observed floating on the surface of water.

### Example 34

Mutation and X-ray Tests. Into a plastic 5-gallon white bucket were place 1 kilogram of 99.9 silver shots of about 1 mm to 10 mm, 1 kilogram of Fleischmann's instant dry yeast and 1 gallon of Arrowhead Mountain Spring Water. The mixture was thoroughly mixed with a wooden spoon and the bucket was placed in an electric kiln heated a temperature of 43° C. (109° F.). The contents of the bucket were stirred about every 12 hours. After 4 days, the silver shots were a light yellow color. After eight days, the yeast mixture was decanted into another bucket and the silver shots were thoroughly washed with water. The silver shots were now a yellow color. About 10% of the silver shots were a pale bluish color and about 10% of the silver shots were a light copper color.

### Example 34A

An Oxford 2000 X-ray fluorescence scan of the surface of a silver shot recovered after 8 days of mutation in Example 34 showed about 0.001 percent gold.

### Example 35

Mutation in Electromagnetic Field. A 2-liter beaker was tightly wrapped with 125 feet of 14 gauge insulated copper wire. One end of the copper wire was connected to the white wire and the other end was connected to the black wire of a two wire 12 gauge extension cord. The cord plugged into a Superior Electric variable transformer. The beaker was filled with 100 grams of 99.9% casting silver granules of 1 mm to 10 mm, 100 grams of Saccharomyces cerevisiae (Fleischmann brand) and 1000 ml of distilled water. Transformer was adjusted for 7 to 7.5 amps of current through the copper wire to create an electromagnetic field in the beaker. The temperature of the microbial solution varied from about 35° C. to 39° C. An air pump and air stone was used to agitate and to provide air to the microbial solution. Distilled water was added as needed to maintain the microbial solution at about 1000 ml. After a few days, the microbe density was in the range of 1% to 3% by weight and the silver granules were coated with a thin layer of a yellow material.

### Example 36

Precious Metal Production in Resonating Aluminum Tubing. A 14-feet length of Anderson Barrows ⅜ inch soft aluminum coil tubing purchased at Ace Hardware was coiled in loops of about 2.5 to 3 inch diameter. The coiled tubing was place into a 2-liter beaker was tightly wrapped with 125 feet of 14 gauge insulated copper wire. The ends of the copper wire were connected to a two wire extension cord and the cord was plugged into a Superior Electric variable transformer. Transformer was adjusted for 7 to 7.5 amps of current through the copper wire to create a magnetic field in the beaker. The air temperature in the beaker at the center of coiled aluminum tube varied from about 30° C. to 35° C. An air pump was used to transfer the air flow from the resonating aluminum tubing to a 2 liter beaker filled with 1 liter of distilled water and 20 grams of S. Cerevisiae (Fleischmann's yeast). After 24 hours, S. Cerevisiae solution was decanted from the beaker to give 3.9 grams of a black metal powder. The metal production was continued for six days. A total of 24 grams of black metal was produced. The black metal was separated and dried. A 7.0 gram sample of the black metal was wrapped in 10 grams of assay-grade lead sheet, placed in a bone ash cupel and heated in an electric kiln heated to 500° C. The temperature was gradually increased to 1000 C during a 1 hour period and then maintained at 1000° C. for 30 minutes. The cupel was removed from the kiln and cooled. A silvery precious metal bead weighing 6.4 grams was produced.

### Example 37

Precious Metal Production in Aluminum Tubing with Internal Electrical Circuit. A 10-feet length of Anderson Barrows ⅜ inch soft aluminum coil tubing purchased at Ace Hardware was coiled in loops of about 2.5 to 3 inch diameter. Alligator clips were added near to the ends of a two wire 12 gauge cooper extension cord. The extension cord was plugged into a Superior Electric variable transformer and one alligator clip was connected to each end of the aluminum tubing. Transformer was adjusted for 5 to 6 amps of AC current through aluminum tubing to create an electromagnetic field inside the aluminum tubing. The air temperature at the center axis of coiled aluminum tubing varied from about 30° C. to 35° C. An air pump was used to transfer the air flow from the resonating aluminum tubing to a 2 liter beaker filled with 1 liter of distilled water and 20 grams of S. Cerevisiae (Fleischmann's yeast). After 24 hours, S. Cerevisiae solution was decanted from the beaker to give 3.1 grams of a white metallic metal. The metal production was continued for 3 days. A total of 9.0 grams of off-white metal was produced. The off-white metal was separated and dried. A 4.0 gram sample of the off-white metal was wrapped in 6 grams of assay-grade lead sheet, placed in a bone ash cupel and heated in an electric kiln heated to 500° C. The temperature was gradually increased to 1000 C during a 1 hour period and then maintained at 1000° C. for 30 minutes. The cupel was removed from the kiln and cooled. A silvery precious metal bead weighing 3.6 grams was produced.

The aluminum tubing in Example 37 was weighed before and after six days of operation as described in Example 37 on an electronic balance with an accuracy of 0.1 milligram. No weigh change was detected.

Methods for producing mutant microbes that coat silver with a yellow metal and uses of the mutant microbes for producing and recovering precious metal and producing biofuels and oil products have been described in the accordance with the embodiments shown, and one of ordinary skill in the art will readily recognize that there could be variations to the embodiments, and any variation would be within the spirit and scope of the present invention. Accordingly, many modifications may be made by one of ordinary skill in the art without departing from the spirit and scope of the appended claims.

What is claimed is:

1. A mutant microbe used for generating trace amounts of gold particles on metallic silver, the mutant microbe produced by placing metallic silver in an aqueous solution and adding a species of Saccharomyces to the aqueous solution such that when the species of Saccharomyces comes in contact with the metallic silver, a yellow layer comprising a trace amount of nano gold particles forms on the metallic silver and at least a portion of the species of Saccharomyces transforms into the mutant microbe.

2. The mutant microbe of claim 1 wherein air flow is provided to the aqueous solution from one of a resonating aluminum tube and a resonating silver tube in an electromagnetic field.

3. The mutant microbe of claim 1 wherein the species is Saccharomyces cerevisiae.

4. A method of producing a mutant microbe used for generating trace amounts of gold particles on metallic silver, the method comprising: placing metallic silver in an aqueous solution and adding a species of Saccharomyces to the aqueous solution such that when the species of Saccharomyces comes in contact with the metallic silver, at least a portion of the species of Saccharomyces transforms into a mutant microbe that interacts with the metallic silver to form a yellow layer comprising a trace amount of nano gold particles on the metallic silver.

5. The method of claim 4 further including providing air flow to the aqueous solution from one of a resonating aluminum tube and a resonating silver tube in an electromagnetic field.

6. The method of claim 4 in which the species is Saccharomyces cerevisiae.

7. The method of claim 4 wherein clusters of precious metals are formed in the cytoplasm of the mutant microbe and the clusters of precious metals are recovered from the mutant microbes.

8. The method of claim 4 wherein clusters of precious metals are formed in the aqueous solution and the clusters of precious metals are recovered from the aqueous solution.

9. A method of recovering precious metals from a mineral ore, the method comprising: placing metallic silver in an aqueous solution and adding a species of Saccharomyces to the aqueous solution such that when the species of Saccharomyces comes in contact with the metallic silver, at least a portion of the species of Saccharomyces transforms into a mutant microbe that interacts with the metallic silver and forms a yellow and copper colored layer comprising a trace amount of nano gold particles on the metallic silver; and contacting a mineral ore with the aqueous solution including the mutant microbe.

10. The method of claim 9 further comprising providing air flow to the aqueous solution from one of a resonating aluminum tube and a resonating silver tube in an electromagnetic field.

11. The method of claim 9 wherein the species is Saccharomyces cerevisiae.

12. A method of producing oil products from at least one of a sedimentary organic rock, heavy oil and a biomass, the method comprising: placing metallic silver in an aqueous solution and adding a species of Saccharomyces to the aqueous solution such that the when the species of Saccharomyces comes in contact with the metallic silver, at least a portion of the species of Saccharomyces transforms into a mutant microbe that interacts with the metallic silver and forms a yellow to copper colored layer comprising a trace amount of nano gold particles on the metallic silver; and contacting at least one of the sedimentary organic rock, the heavy oil and the biomass with the mutant microbe.

13. The method of claim 12 wherein the sedimentary organic rock includes at least one of oil shale and oil sands, and wherein air flow is provided to the aqueous solution from one of a resonating aluminum tube and a resonating silver tube in an electromagnetic field.

14. The method of claim 12 wherein the species is Saccharomyces cerevisiae

15. The method of claim 12 wherein the biomass includes dead mutant microbes and wherein air flow is provided to the aqueous solution from one of a resonating aluminum tube and a resonating silver tube in an electromagnetic field.

16. The method of claim 12 in which the species is Saccharomyces cerevisiae.

17. A method of bioconverting heavy oil to lower viscosity oil, the method comprising: placing metallic silver in an aqueous solution and adding a species of Saccharomyces to the aqueous solution such that when the species of Saccharomyces comes in contact with the metallic silver, at least a portion of the species of Saccharomyces transforms into a mutant microbe and a layer comprising a trace amount of nano gold particles forms on the metallic silver; and contacting the heavy oil with the mutant microbe.

18. The method of claim 17 wherein the metallic silver is from 1 micrometer particles to silver bars and wherein air flow is provided to the aqueous solution from one of a resonating aluminum tube and a resonating silver tube in an electromagnetic field.

19. A method of producing nano atoms of precious metals, the method comprising resonating one of an aluminum tube and a silver tube in an electromagnetic field.

20. The method of claim 20 wherein the nano atoms are aggregated into clusters of bulk precious metals with a biodegradable organic medium.
